(19) United States
(12) Patent Application Publication
(10) Pub. No.: US 2017/0274119 A1
(43) Pub. Date: Sep. 28, 2017

(54) MOULDING FOR REPLICATING A STRUCTURE OF A BIOLOGICAL TISSUE AND METHOD FOR PRODUCING THE SAME

(71) Applicant: Technische Universitaet Ilmenau, Ilmenau (DE)

(72) Inventors: Andreas Schober, Erfurt (DE); Joerg Hampl, Erfurt (DE); Sebastian Haefner, Dresden (DE); Justyna Tobola, Ilmenau (DE); Frank Weise, Ilmenau (DE); Sukhdeep Singh, Ilmenau (DE); Gregor Schlingloff, Ilmenau (DE)

(21) Appl. No.: 15/508,565

(22) PCT Filed: Aug. 26, 2015
(86) PCT No.: PCT/EP2015/069522

§ 371 Date: Mar. 3, 2017

(30) Foreign Application Priority Data
Sep. 3, 2014 (DE) 10 2014 112 660.2

(51) Int. Cl. A61L 27/16; A61L 27/50; H01L 29/06; A61L 29/14; A61L 31/14; A61B 17/34; A61L 29/08

(52) U.S. Cl. CPC A61L 27/16; A61B 17/3417; A61L 27/50; A61L 29/085; A61L 29/14; A61L 31/14; H01L 29/06; A61B 2017/00849

(57) ABSTRACT

A method for replicating a structure of a biological tissue provides a plastically deformable film that is subjected to a pressure in order to press it into a mold. The mold comprises formations for pit-like depressions, recesses and notches. The recesses each border on at least one of the pit-like depressions and are opened up. The notches form at least one film hinge in the film. The shaped film is folded into a stack having at least two layers of film, the film hinge forming the folding edge for the folding process. The pit-like depressions are closed along their direction of extension by a neighboring layer of the stack and form each time a capillary. At least two of the opened recesses are arranged one on top of another and form a canal arranged perpendicular to the plane of extension of the film.

### FIELD

The present invention concerns first of all a method for producing a molding for replicating a structure of a biological tissue. The molding serves in particular for colonization of cells and comprises corresponding vessels, so that for example a liver or a blood-brain barrier can be replicated. Moreover, the invention concerns a molding for replicating a structure of a biological tissue.

### BACKGROUND

Biological systems, in particular also organisms such as animals and humans possess various organs which consist of different cell types and which have a well-defined morphology, despite a certain variance among different individuals. A key important element here is vascularization, i.e., the supplying of the tissue with the aid of blood capillaries. The different functionality of the individual organ is also manifested in the different structure or in the different properties of the blood capillaries. In the liver, the endothelium of the blood capillaries is constructed such that a certain permeability is realized in the space of Disse, while in the brain the blood-brain barrier is constructed such that a direct passage of substances from the blood to the tissue is not possible, since endothelial cells and pericytes form an impenetrable barrier. One example is the stroma of bone marrow, which is replete with numerous thin-walled blood vessels which are known as the bone marrow sinus. The wall of the sinusoids is formed by a delicate, irregularly perforated endothelium having no basal lamina. The capillary density in the brain, especially in the gray matter of the cerebral cortex, is very high. The mean distance between individual capillaries is only 40 μm against a diameter of 3 μm to 7 μm. Liver sinusoids are capillaries with a length of around 0.5 mm and a diameter of 9 μm to 12 μm.

WO 2012/045687 A1 reveals a structure for replicating a small blood vessel in the form of a sinusoid, such as occurs in the liver. Each time an intermediate space is formed between several layers of a porous material arranged one on top of another. The layers comprise a co-culture of cell species present in the sinusoid being replicated. Furthermore, canals are formed in the layers which connect the intermediate spaces and are designed to transport a fluid such as blood.

WO 02/053193 A2 teaches a multilayered structure with a first layer consisting of a material suitable for colonization by animal cells. The first layer comprises a pattern of microcanals, the canals being suited to the colonization of animal cells and to the circulation of fluid through the layer. Furthermore, the structure comprises at least one second layer of a material suitable for the colonization of animal cells. The first and second layer are joined together, thereby creating canals. The first layer can serve for the colonization of endothelial cells and the second layer for the colonization of epithelial cells such as hepatocytes. The layers can be stacked one on top of another and are joined together by through holes.

From WO 2004/026115 A2 a method is known for producing a structure for the replicating of a sinusoid possessing several two-dimensional layers stacked one on top of another, on which endothelial cells and hepatocytes are arranged, for example. The layers can have through holes.

The problem which the present invention proposes to solve is to carry out the formation of vessels during the producing of structures serving for replicating of a biological tissue in a lower-cost and more precise manner.

This problem is solved by a method according to the enclosed claim 1 as well as by a molding according to the enclosed subsidiary claim 10.

### SUMMARY

The method according to the invention serves to produce a molding for the replicating of a structure of a biological tissue. The molding replicates a three-dimensional structure of the biological tissue and serves in particular to accommodate biological cells and liquids. The biological cells are colonized in the molding and preferably form a component of the molding. The molding enables an exchange of materials, especially an exchange of liquids between the cells and the outside of the molding. The biological tissue being replicated is preferably formed by a liver or liver structures, by a blood-brain barrier, by a bone marrow structure or by a muscle or muscle structure. Yet other kinds of biological tissue can be replicated, such as kidney tissue or spleen tissue. The biological tissue can be human, animal, or plant.

In one step of the method according to the invention, a plastically deformable film is provided. The film consists preferably of a biocompatible material, especially preferably of a biocompatible plastic.

In a next step, the film is subjected to a pressure in order to press it into a mold. The mold comprises formations for pit-like depressions, for recesses and for notches. Thus, pit-like depressions, recesses and notches are formed in the film when it is pressed into the mold. The pit-like depressions are formed in the plane of extension of the film, so that the pit-like depressions extend along the plane of extension of the film. The recesses each border on at least one of the pit-like depressions. The recesses each have a bottom, at which they are opened, so that each time an opening is produced. In this, at least lateral portions of the recess shape are preserved, so that they are recesses open on top and on the bottom. The notches form at least one film hinge in the film, at which the film can be folded.

In a further step, a folding of the formed film is done to produce a stack comprising at least two layers. The film hinge here forms the folding edge for the folding. Thus, at least one folding process occurs, during which the film is pivoted at the film hinge by 180°, so that two portions of the film come to lie on each other and form the layers of the stack. The layers have preferably the same length and width and are arranged congruently one on top of another in the stack. Preferably several folding steps are done, for which a corresponding number of film hinges are formed in the film. The folding is done preferably in Leporello fashion, i.e., a zig zag alternating once forward and once backward. But other folding procedures can also be chosen, for example, by forming partial stacks which are in turn folded on each other, the individual partial stacks being folded in a different direction than the partial stacks to each other. As a result of the folding, the pit-like depressions located in one of the layers are closed along their direction of extension by the neighboring layers of the stack, so that they form a capillary. The capillaries extend in the same way as the pit-like depressions in the plane of extension of the film. The capillaries preferably form blood capillaries or guiding canals for the growth of neurites. As a result of the folding, at least two of the opened recesses are arranged one on top of the other, so that they form a canal arranged perpendicular to the plane of extension of the film. Since the recesses are each connected to at least one of the pit-like depressions, the canals formed by the recesses are also connected each to at least one of the capillaries, so that liquid can flow from the canals into the capillaries and back again. The folded stack forms the molding being produced. The molding is preferably placed in a bioreactor system, for which connections to the canals need to be created. Preferably, several moldings are stacked one on top of the other and oriented to form a complex system for the replicating of biological structures.

A particular benefit of the method according to the invention is that the film hinges enables an exact and low-cost arranging of the film portions one on top of another, so that the vessels being formed, i.e., the capillaries and canals being formed, are formed according to specification. The film hinges are formed with the same accuracy as the pit-like depressions and the recesses, so that a precisely fitted placement one on top of another is assured. No costly positioning is needed. In particular, the method according to the invention allows the capillaries and the canals arranged perpendicular to them to be formed at the same time, as is needed in order to replicate many biological structures.

In preferred embodiments of the method according to the invention the film is heated prior to the pressing. Insofar as the film consists for example of polycarbonate or a comparable material, it is preferably heated to a temperature between 140° C. and 180° C., so that it has this temperature during the pressing. Insofar as the film consists of a biodegradable polymer, for example, it is heated preferably to a temperature between 35° C. and 80° C. The heating is done preferably via the mold, i.e., the film is indirectly heated via the mold before pressure is applied to it.

The mold can consist of a hard or also a conditionally flexible material. It consists preferably of silicon or polydimethylsiloxane (PDMS). The mold can be formed as a positive mold or a negative mold, i.e., the formations for the pit-like depressions, for the recesses and for the notches can be raised or sunken.

In especially preferred embodiments of the method according to the invention, polycarbonate (PC) is used as the material for the film. In alternative preferred embodiments, cyclo-olefin copolymer (COC), styrene-acylnitrile (SAN) or polystyrene (PS) is used as the material for the film. In further alternative preferred embodiments, a biodegradable polymer such as polylactic acid is used as the material for the film.

The capillaries and the canals are arranged in the molding being produced preferably in the same way as in the tissue being replicated. Therefore, the formations of the mold are created according to the tissue being replicated, which can be done for example by lithographic methods or by fabrication of the mold by machining.

The pressure is preferably transmitted to the film with the aid of a gas.

In simple embodiments of the method according to the invention, during the folding the pit-like depressions of the one layer are closed along their direction of extension by flat portions of the layer situated above them. In preferred embodiments, however, two of the pit-like depressions from the two layers meet each other. As a result of the folding, each time one of the pit-like depressions of one of the layers is arranged congruently across one of the pit-like depressions of the layer located above it, so that the capillary formed between them is symmetrically formed between the two layers. The pit-like depressions in this case preferably have the shape of a hollow half-cylinder, so that the capillary formed has approximately the shape of a hollow cylinder.

The recesses can be opened up in various ways. In a first preferred embodiment, the recesses are opened up at their bottom in that they are molded far enough into the film that the film rips at the bottoms of the recesses. Due to the forming process, the film becomes increasingly thin in the area of the bottoms, until it rips open. In a second preferred embodiment, the recesses are opened up at their bottoms by dissolving the bottoms of the recesses in an etching bath. For example, the shaped film can be dipped into the etching bath so that only the bottoms of the recesses find themselves in the etching bath and are thereby dissolved.

In especially preferred embodiments, prior to the folding of the shaped film biological cells are arranged at least on one of the two sides of the shaped film. Thus, the biological cells after the folding already find themselves in cavities between the layers of the stack, for example, in the capillaries. In this way, the biological cells can be introduced into these cavities at low cost. For the most part, the relatively simple folding procedure does not hinder the colonization of the biological cells. Preferably, the biological cells are arranged on both sides of the shaped film before its folding. Thus, the biological cells can be introduced into the capillaries and also into other cavities between the layers of the stack; preferably, by the introduction of a cell suspension through capillary forces. Preferably different kinds of biological cells are placed on the two sides of the shaped film in order to replicate complex biological tissue. The biological cells can be formed, for example, by hepatocytes, endothelial cells, Kupffer cells or stellate cells.

In addition, after the folding, other biological cells are preferably introduced into the stack, i.e., into the molding. For example, the biological cells can be pumped or flushed into the canals and/or into the capillaries.

In further especially preferred embodiments of the method according to the invention, prior to the shaping of the film at least one layer of a cell attractant or a cell repellent is applied to the formations of the mold. The cell attractant or cell repellent is applied to the film during the shaping of the film such that areas are defined on the shaped film which either attract or repel the biological cells. It has been convincingly demonstrated that the cell attractant or the cell repellent can also be applied to the film via the mold in the heated state of the film during its shaping, which affords the special advantage that the cell attractant or the cell repellent is applied to the film in accordance with the structure of the formations. In this way, the cell attractant or cell repellent can be applied to the film at low cost and with high accuracy. Since the mold contains the formations for the pit-like depressions and recesses, the cell attractant or cell repellent is preferably applied such that—depending on whether it is a positive mold or a negative mold—either only the formations or only the areas of the mold outside the formations are provided with the cell attractant or the cell repellent. Thus, for example, one can ensure that the cell attractant only gets into the pit-like depressions, i.e., into the capillaries, so that the biological cells are only colonized there. The mold thus forms a punch, for which the mold consists of a flexible or rigid material, such as PDMS or silicon.

Alternatively or additionally, preferably after the molding of the film a layer of a cell attractant or a cell repellent is applied to the molded film; preferably either only in the pit-like depressions and/or in the recesses or only in the areas outside of the pit-like depressions and/or the recesses.

The cell attractant is preferably formed by a hydrogel, such as collagen hydrogel. The cell repellent is preferably formed by a hydrogel, such as poly-NIPAAm-hydrogel.

The film provided has a thickness of preferably between 1 μm and 1 mm, more preferably between 10 μm and 200 μm. In especially preferred embodiments of the method according to the invention, the thickness of the film provided is between 20 μm and 80 μm.

The film provided has a width and a length between 1 cm and 50 cm, especially preferably between 5 cm and 20 cm.

The film preferably has pores through which substances, especially liquids, can get to or from the biological cells. The pores in the film provided have a diameter preferably between 10 nm and 10 μm. In especially preferred embodiments, the pores in the provided film have a diameter between 100 nm and 5 μm, more preferably between 500 nm and 2 μm.

The density of the pores in the film, i.e., the number of pores per unit of surface of the provided film, is preferably at least 10⁵ pores per cm², especially preferably at least 10⁶ pores per cm². The density of the pores in the provided film is preferably at most 10⁷ pores per cm².

Electrodes and/or transistor structures are preferably arranged in the film, in order to measure the electrical activity of the biological cells in the subsequent molding.

In preferred embodiments of the method according to the invention, the film during the pressing process is subjected to the pressure, especially by the gas possessing the pressure, indirectly through a sacrifice film.

The molded film is preferably trimmed to remove unwanted edges prior to the folding.

The sacrifice film provided has a thickness of preferably between 1 μm and 500 μm, especially preferably between 10 μm and 100 μm. In especially preferred embodiments of the method according to the invention, the sacrifice film provided has a thickness between 20 μm and 80 μm.

The sacrifice film consists preferably of a plastic, such as a fluorocarbon or silicone. Especially preferably, the sacrifice film consists of perfluorethylene propylene (FEP).

The pit-like depressions have a width preferably between 1 μm and 1 mm; more preferably between 10 μm and 300 μm and especially preferably between 50 μm and 150 μm.

The recesses have a diameter preferably between 10 μm and 5 mm; especially preferably between 100 μm and 1 mm.

The pit-like depressions and the recesses or the capillaries and canals formed from them preferably have the same arrangements for the replication of the biological tissue as are also present in the biological tissue being replicated. In the case of a liver structure being replicated, each time several of the pit-like formations extend preferably in the shape of a star from one of the opened recesses in the plane of extension of the film. Accordingly, each time several of the capillaries preferably extend in the shape of a star from one of the canals in the plane of extension of the respective layer of the stack. The pit-like formations are also preferably arranged in the form of a triangular grid in the plane of extension of the film. Accordingly, the capillaries are preferably arranged in the form of a triangular grid in the plane of extension of the respective layer of the stack. The triangular grid preferably forms a hexagonal grid. Preferably each time one of the recesses forms a point of intersection of the pit-like formations. Accordingly, each time one of the canals lies preferably at the point of intersection of the capillaries.

The molding according to the invention can be produced with the method according to the invention.

The molding according to the invention serves to replicate a structure of a biological tissue. The molding is used preferably to replicate a structure of a liver or a liver structure, such as liver sinusoids and hepatic lobules, a blood-brain barrier, or a muscle, while in principle many other kinds of biological tissues can also be replicated.

The molding according to the invention comprises a film which is fashioned in the form of a stack of at least two layers of the film. Thus, there are portions of film arranged one on top of another. The film comprises pit-like depressions. The pit-like depressions extend in the direction of extension of the film, i.e., perpendicular to the stack direction. The pit-like depressions are closed along their direction of extension by the neighboring layer of the stack, so that capillaries are formed. Thus, the neighboring layer, i.e., the layer located immediately above or below in the stack, forms a cover for the respective pit-like depression, so that the respective pit is closed at the top or bottom and forms the capillary. The capillaries are preferably open at their axial ends. The film furthermore comprises recesses, which are open at their bottoms, so that substances can get through the recesses. Each time, several of the recesses are arranged one on top of another within the stack, so that these form a canal arranged perpendicular to the plane of extension of the film. Thus, the one canal or the several canals extend perpendicular to the capillaries. The one canal or the several canals each time border on at least one of the capillaries, so that substances can flow from the respective canal into the adjoining capillary and back again. The layers of the stack are connected in pairs at one edge of the stack by a film hinge which is formed in the film. Thus, the stack is preferably formed by a single film. This single film has been folded to form the stack, the layers of the stack being formed by individual portions of the single film.

The molding according to the invention preferably also has those features which are indicated in connection with the method according to the invention and its preferred embodiments.

Biological cells are preferably arranged in the molding according to the invention. These cells are able to exchange materials via the capillaries and via the canals, so that the cells can survive and perform their specific functions. The film is preferably porous, so that the biological cells are furthermore able to exchange substances via the pores.

Preferably, the biological cells of a first kind are colonized between the layers of the stack and/or on the top side and/or on the bottom side of the stack and the biological cells of a second kind are colonized in the capillaries. In this way, complex biological tissues can be replicated, such as hepatic lobules. In this case, the biological cells of the first kind are formed preferably by hepatocytes, while the biological cells of the second kind are formed preferably by endothelial cells.

Preferably, a cell attractant or a cell repellent layer on the film is present solely in the capillaries and/or in the canals. Additionally or alternatively, a cell attractant or a cell repellent layer on the film is present solely in areas outside of the capillaries and/or the canals.

### BRIEF DESCRIPTION OF THE DRAWINGS

Further benefits, details, and modifications of the invention will emerge from the following description of preferred embodiments of the invention, making reference to the drawing. There are shown:

FIG. 1 is a method for pressing a film into a mold according to one preferred embodiment of the method according to the invention;

FIG. 2 is the film shown in FIG. 1 in a modified embodiment after the molding process;

FIG. 3 is the film shown in FIG. 2 prior to the folding process according to a first preferred embodiment;

Fig. : is the film shown in FIG. 3 in the folded state;

FIG. 5 is the film shown in FIG. 2 prior to a folding process according to a second preferred embodiment;

FIG. 6 is the film shown in FIG. 5 in the folded state;

FIG. 7 is the film shown in FIG. 4 with biological cells introduced;

FIG. 8 is the film shown in FIG. 1 according to another preferred embodiment;

FIG. 9 is the placement of a cell attractant on the mold shown in FIG. 8;

FIG. 10 is a liver structure being replicated by the method according to the invention;

FIG. 11 is a sinusoid of a hepatic lobule being replicated by the method according to the invention;

FIG. 12 is the replication shown in FIG. 11 in seven-fold iteration;

FIG. 13 is a punch used for the replication shown in FIG. 12;

FIG. 14 is another sinusoid of a hepatic lobule replicated by the method according to the invention;

FIG. 15 is the replication shown in FIG. 11 in seven-fold iteration; and

FIG. 16 is a punch used for the replication shown in FIG. 15.

### DETAILED DESCRIPTION

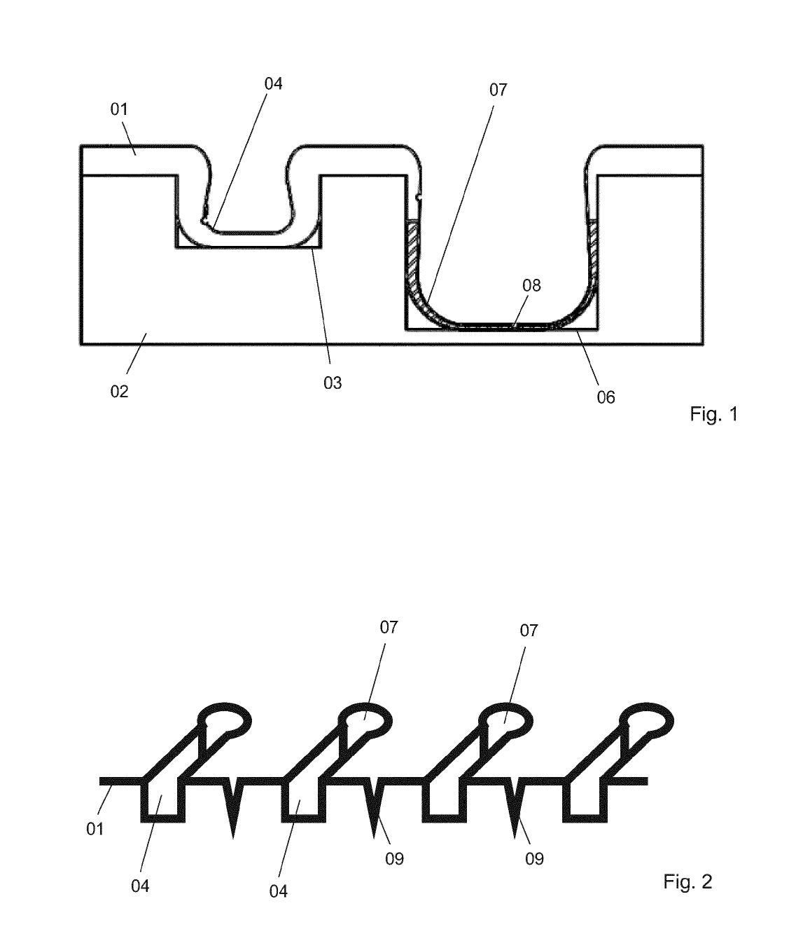

FIG. 1 shows one step of a preferred embodiment of the method according to the invention. During this step, a thermoplastic film 01 is pressed with the aid of a forming gas (not shown) into a mold 02 fashioned as a negative mold. The mold 02 has a formation 03 for a pit-like depression, so that a pit-like depression 04 is produced in the film 01. Furthermore, the mold has a deeper formation 06 for a recess, so that a recess 07 is produced in the film 01. The film 01 is pressed far enough into the recess 06 until the film 01 rips open in the area of a bottom 08 of the recess 07. Furthermore, the mold has formations (not shown) for notches, so that several notches 09 (shown in FIG. 2) are produced in the film 01.

FIG. 2 shows the film m01 shown in FIG. 1 in a modified embodiment after the molding process in a partial perspective view. The modification consists in the arrangement of the pit-like depressions 04, the recesses 07 and the notches 09.

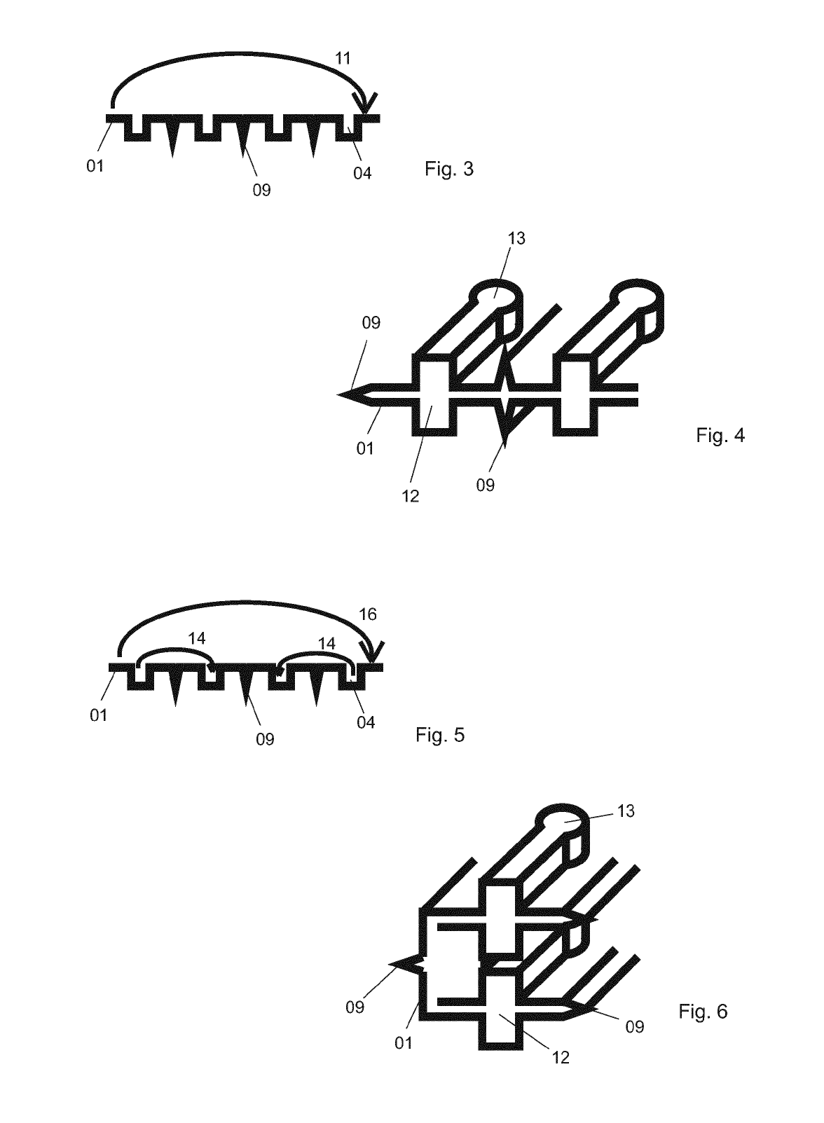

FIG. 3 shows the film 01 shown in FIG. 2 prior to a folding process according to a first preferred embodiment. The folding process being carried out is symbolized by an arrow 11. The folding is done at the central notch 09, which functions here as a film hinge.

FIG. 4 shows the film 01 shown in FIG. 3 in the folded state. One half of the film 01 has been pivoted by 180° about the central notch 09, so that this meets the other half of the film 01 in congruent manner. In this way, the pit-like depressions 04 are brought together at their long sides, so that they form a capillary 12 pairwise each time. At the same time, the open recesses 09 have been placed one on top of another, so that they form a canal 13 pairwise each time, which extends perpendicular to the capillaries 12 and to the film 01. Two of the notches 09 remain unused in this embodiment. The folded film 01 forms the molding being produced according to the invention.

FIG. 5 shows the film 01 shown in FIG. 2 prior to a folding process according to a second preferred embodiment. The folding process being done involves a first step, which is symbolized by two arrows 14, and a second step, which is symbolized by an arrow 16. The folding is done in the first step at the two outer notches 09, each of which acts as a film hinge, and in the second step it is done at the central notch 09, which then acts likewise as a film hinge.

FIG. 6 shows the film 01 shown in FIG. 5 in the folded state. The two outer quarters of the film 01 have been pivoted in a first step 14 (shown in FIG. 5) by 180° about the two outer notches 09 and 180° inwardly, so that these meet the two inner quarters of the film 01 in congruent manner. In this way, the pit-like depressions 04 are joined in pairs along their long sides, so that they each form one of the capillaries 12. At the same time, the open recesses 09 have been placed one on top of another in pairs, so that they form part of the canal 13, which extends perpendicular to the capillaries 12. In the second step 16 (shown in FIG. 5), the two already folded halves of the film 01 have been pivoted by 180° about the inner notches 09, so that these meet each other in congruent manner. In this way, the already formed parts of the canal 13 are placed one on top of another, so that the canal 13 is completely formed.

None of the notches 09 remained unused in this embodiment. The folded film 01 forms the molding being produced according to the invention.

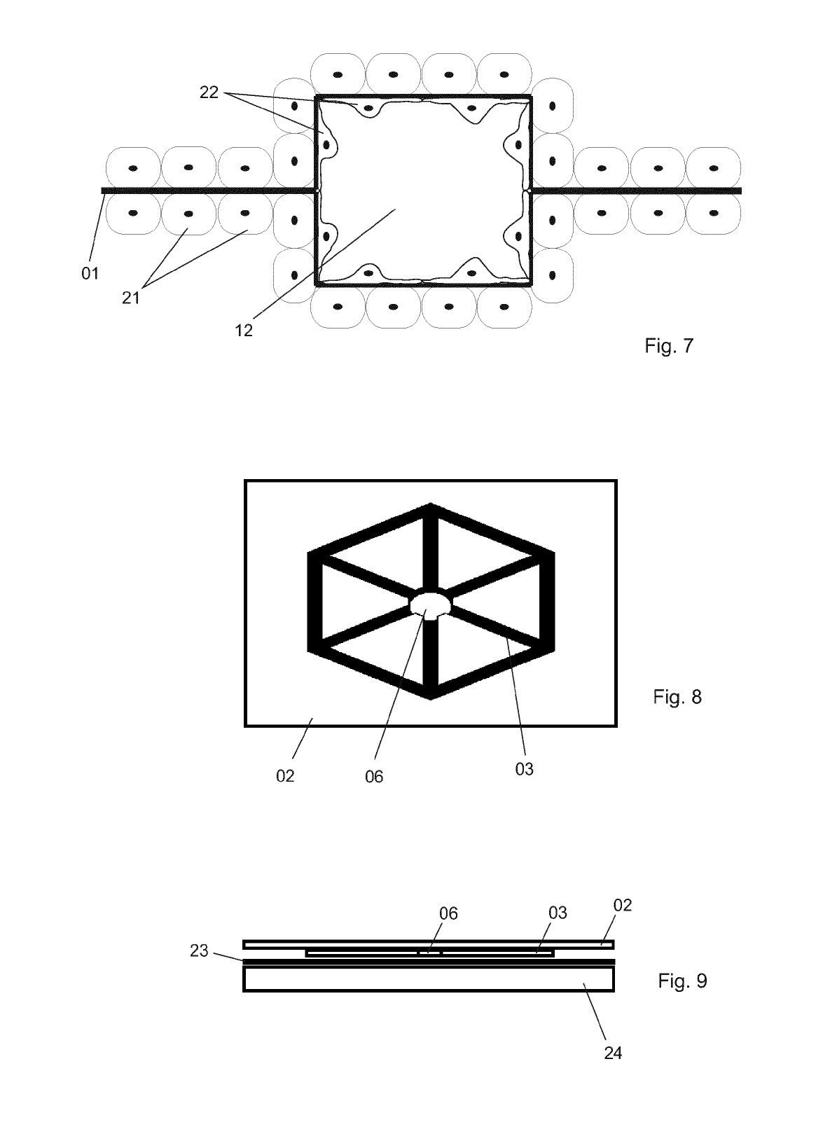

FIG. 7 shows the folded film 01 shown in FIG. 4, i.e., the molding being produced according to the invention in a detail view, where in particular one of the capillaries 12 is shown in cross section. This embodiment of the molding according to the invention serves to replicate liver tissue, for which biological cells, namely hepatocytes 21 and endothelial cells 22, have been introduced into the molding. The endothelial cells 22 have been introduced into the capillary 12, while the hepatocytes 21 were colonized in the areas outside of the capillary 12. The colonization of the biological cells 21, 22 is preferably controlled by a cell attractant 23 (shown in FIG. 9).

FIG. 8 shows the mold 02 shown in FIG. 1 in a modified preferred embodiment, which is suitable as a positive mold especially for the producing of the molding to replicate liver tissue. For this, the mold 02 comprises hexagonally arranged raised formations 03 for the pit-like depressions, at whose center the raised formation 06 for the recess is arranged. Thus, the mold 02 configured as a positive mold constitutes a punch.

FIG. 9 shows the placement of the cell attractant 23 on the mold 02 shown in FIG. 8. For this, the cell attractant 23 is at first arranged on a backing 24, such as one of PDMS or glass. Next, the mold 02 with the raised formations 03, 06 is pressed with a slight pressure against the cell attractant 23, whereby the latter comes to adhere to the raised formations 03, 06. During the subsequent pressing of the film 01 into the mold 02 (shown in FIG. 1), which in the case of the mold 02 shown here and configured as a positive mold can be done by pressing the mold 02 against the film 01, once again the cell attractant 23 is placed in the pit-like depressions 04 and in the recesses 07 of the film 01 (shown in FIG. 1). The areas outside of the pit-like depressions 04 and the recesses 07 are not provided with cell attractant 23 in this process.

Instead of the mold 02, as an alternative the molded film 01 can also be pressed directly onto the cell attractant 23 on the backing 24. Instead of the cell attractant 23, a cell repellant can also be applied in the described manner to the mold 02 or the molded film 01.

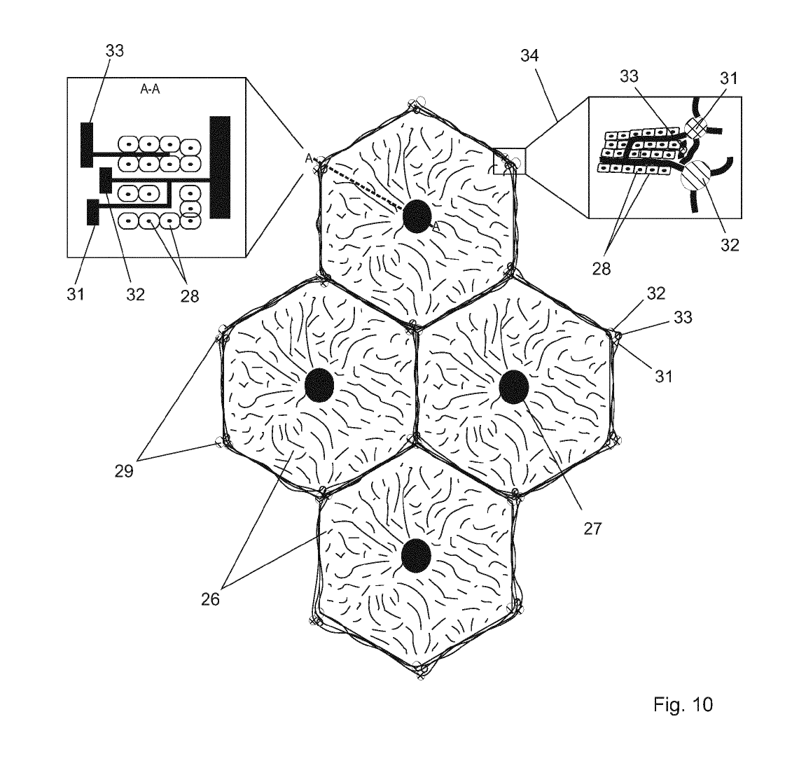

FIG. 10 shows a liver structure being replicated according to the method of the invention. The structure comprises four hexagonally shaped hepatic lobules 26, at whose centers there is situated a Vena centralis 27 each time. A sectional view A-A represents hepatocytes 28 in particular. At the outer corners of the hepatic lobules 26 is found a Glisson trias 29 with an Arteria interlobularis 31, a Vena interlobularis 32 and a Doctuli interlobularis 33, which are shown in particular in a detail representation 34 of one of the Glisson trias 29.

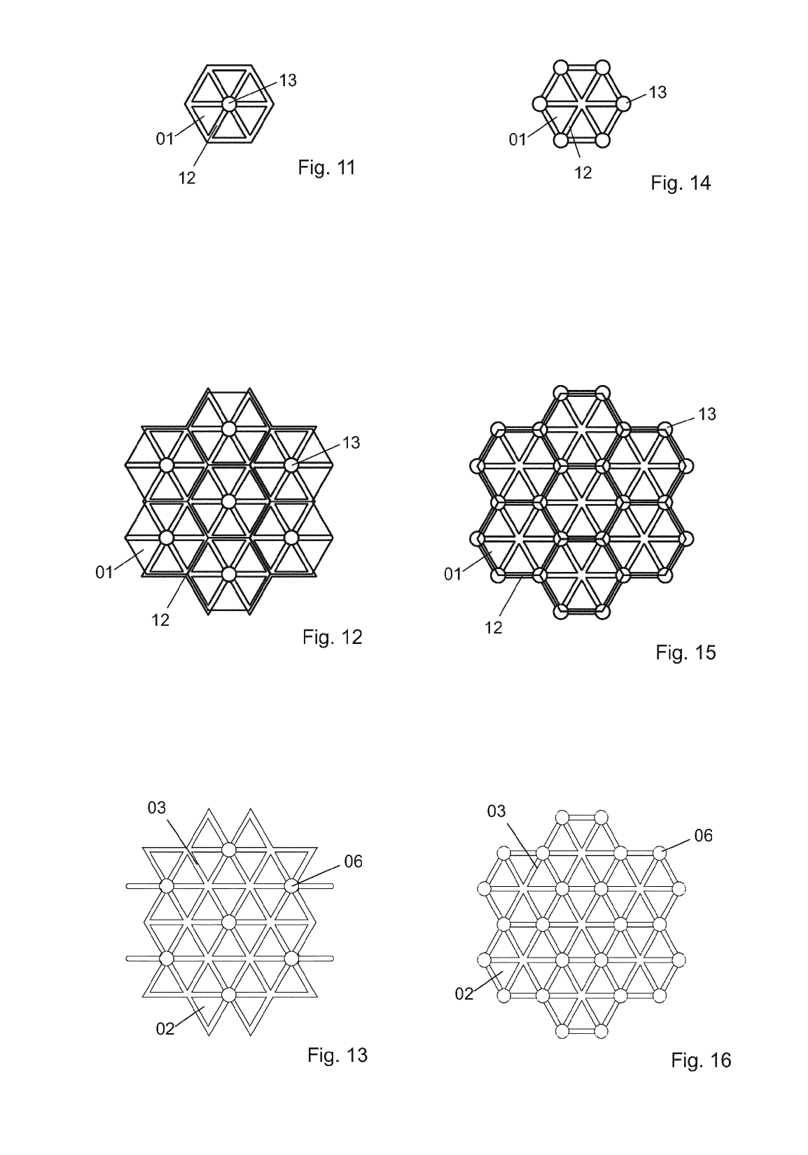

FIG. 11 shows a sinusoid of one of the hepatic lobules 26 (shown in FIG. 10) replicated by the method according to the invention with the canal 13 for replicating the Vena centralis 27 (shown in FIG. 10).

FIG. 12 shows the replication shown in FIG. 11 in a seven-fold iteration in hexagonal arrangement, so that a larger portion of the liver tissue is replicated.

FIG. 13 shows the mold 02 used for the replication represented in FIG. 12, being designed as a punch. The mold 02 comprises the formations 03 for the pit-like depressions and the formations 06 for the recesses.

FIG. 14 shows another sinusoid of one of the hepatic lobules 26 (shown in FIG. 10) replicated by the method according to the invention with the several canals 13 for replicating the Glisson trias 29 (shown in FIG. 10).

FIG. 15 shows the replication shown in FIG. 14 in a seven-fold iteration in hexagonal arrangement, so that a larger portion of the liver tissue is replicated.

FIG. 16 shows the mold 02 used for the replication represented in FIG. 15, being designed as a punch. The mold 02 comprises the formations 03 for the pit-like depressions and the formations 06 for the recesses.

### LIST OF REFERENCE NUMBERS

01 Film

02 Mold

03 Formation for pit-like depression

04 Pit-like depression

05—

06 Formation for recess

07 Recess

08 Bottom

09 Notch

10—

11 Arrow

12 Capillary

13 Canal

14 Arrows

15—

16 Arrow

21 Hepatocytes

22 Endothelial cells

23 Cell attractant

24 Backing

25—

26 Hepatic lobule

27 Vena centralis

28 Hepatocytes

29 Glisson trias

30—

31 Arteria interlobularis

32 Vena interlobularis

33 Doctuli interlobularis

34 Detail

1. A method for producing a molding for replicating a structure of a biological tissue, comprising the following steps:
providing of a plastically deformable film;
subjecting the film to a pressure in order to press it into a mold, the mold comprising formations for pit-like depressions, for recesses and for notches, wherein the pit-like depressions are formed in the plane of extension of the film, while the recesses each border on at least one of the pit-like depressions and each have a bottom, at which they are opened, so that each time an opening is produced, and the notches form at least one film hinge in the film; and
folding of the formed film to produce a stack comprising at least two layers of the film, the film hinge forming the folding edge for the folding, whereby the pit-like depressions are closed along their direction of extension by a neighboring layer of the stack and form a capillary, and whereby at least two of the opened recesses are arranged one on top of the other and form a canal arranged perpendicular to the plane of extension of the film.

2. The method according to claim 1, wherein the film is heated prior to the pressing into the mold to a temperature between 140° C. and 180° C.

3. The method according to claim 1, wherein each time one of the pit-like depressions of one of the layers is arranged congruently across one of the pit-like depressions of the layer located above it, so that the capillary formed between them is symmetrically formed between the two layers.

4. The method according to claim 1, wherein the recesses are opened up at their bottoms in that they are molded far enough into the film that the film rips at the bottoms of the recesses.

5. The method according to claim 1, wherein the recesses are opened up at their bottoms by dissolving the bottoms of the recesses in an etching bath.

6. The method according to claim 1, wherein prior to the folding of the shaped film the following step is carried out:
colonizing of biological cells at least on one of the two sides of the shaped film.

7. The method according to claim 1, wherein prior to the molding of the film the following step is carried out:
applying of a layer of a cell attractant (23) or a cell repellent to the formations of the mold.

8. The method according to claim 1, wherein the film is subjected to the pressure indirectly through a sacrifice film.

9. The method according to claim 1, wherein the provided film has pores with a diameter between 500 nm and 2 μm.

10. A molding for replicating a structure of a biological tissue, comprising a film, which is configured in the form of a stack of at least two layers;
wherein the film has pit-like depressions, which are closed along their direction of extension by a neighboring layer of the stack, so that capillaries are formed;
wherein the film has recesses which are open at their bottoms, while several of the recesses are arranged one on top of another within the stack, so that a canal arranged perpendicular to the plane of extension of the film is formed by the open recesses arranged one on top of another, which borders at least on one of the capillaries; and
wherein the layers are connected in pairs at one edge of the stack by a film hinge which is formed in the film.

11. The method according to claim 2 wherein each time one of the pit-like depressions of one of the layers is arranged congruently across one of the pit-like depressions of the layer located above it, so that the capillary formed between them is symmetrically formed between the two layers.

12. The method according to claim 2, wherein the recesses are opened up at their bottoms in that they are molded far enough into the film that the film rips at the bottoms of the recesses.

13. The method according to claim 2, wherein the recesses are opened up at their bottoms by dissolving the bottoms of the recesses in an etching bath.
